(19) United States
(12) Patent Application Publication
(10) Pub. No.: US 2024/0009394 A1
(43) Pub. Date: Jan. 11, 2024

(54) Insertion Mechanism with Automatic Activation

(71) Applicant: Becton, Dickinson and Company, Franklin Lakes, NJ (US)

(72) Inventors: Danielle Aboud, Dublin (IE); Steve Beguin, Rathdrum (IE)

(21) Appl. No.: 17/860,487

(22) Filed: Jul. 8, 2022

(51) Int. Cl. A61M 5/20; A61M 5/142; A61M 5/31

(52) U.S. Cl. CPC A61M 5/2033; A61M 5/14248; A61M 5/3134; A61M 2005/3132; A61M 2205/02

(57) ABSTRACT

An insertion mechanism for a drug delivery device including a reservoir and a pump configured to deliver fluid from the reservoir includes a fluid path configured to be in fluid communication with the reservoir, an activation member in fluid communication with the fluid path, and an energy storage member connected to the activation member. The energy storage member has a stored state and a released state, where the energy storage member transitions from the stored state to the released state when fluid from the reservoir contacts the activation member.

### BACKGROUND OF THE INVENTION

### Field of the Invention

The present disclosure relates to an insertion mechanism with automatic activation for a drug delivery device.

### Description of Related Art

Wearable medical devices, such as automatic injectors, have the benefit of providing therapy to the patient at a location remote from a clinical facility and/or while being worn discretely under the patient's clothing. The wearable medical device can be applied to the patient's skin and configured to automatically deliver a dose of a pharmaceutical composition within a predetermined time period after applying the wearable medical device to the patient's skin. After the device delivers the pharmaceutical composition to the patient, the patient may subsequently remove and dispose of the device.

### SUMMARY OF THE INVENTION

In one aspect or embodiment, an insertion mechanism for a drug delivery device including a reservoir and a pump configured to deliver fluid from the reservoir includes a fluid path configured to be in fluid communication with the reservoir, an activation member in fluid communication with the fluid path, and an energy storage member connected to the activation member, with the energy storage member having a stored state and a released state. The energy storage member transitions from the stored state to the released state when fluid from the reservoir contacts the activation member.

The activation member may be configured to seal after coming into contact with fluid from the reservoir. The activation member may include a hydrophilic material, with the hydrophilic material having a first tensile strength when dry and a second tensile strength when wet, where the first tensile strength is greater than the second tensile strength. The hydrophilic material may prevent fluid from passing through the activation member once the hydrophilic material is fully saturated by fluid.

The activation member may include a dissolvable material positioned between absorbent materials, with the dissolvable material configured to disintegrate when in contact with a fluid.

The activation member may include a hydrophobic layer and a hydrophilic layer, with the hydrophobic layer and the hydrophilic layer defining a plurality of pores, where the hydrophilic layer is configured to expand closing the plurality of pores when the hydrophilic layer is in contact with a fluid. The energy storage member may be a spring.

In a further aspect or embodiment, a drug delivery device includes a housing, a reservoir positioned within the housing and configured to receive a fluid, a fluid path in fluid communication with the reservoir, a delivery sub-system configured to deliver a fluid from the reservoir to the fluid path, an insertion mechanism comprising a cannula in fluid communication with the fluid path, with the insertion mechanism configured to move the cannula from a retracted position where the cannula is positioned within the housing to an extended position where at least a portion of the cannula is positioned outside of the housing, an activation member in fluid communication with the fluid path, and an energy storage member connected to the activation member. The energy storage member has a stored state when the cannula is in the retracted position and a released state when the cannula is in the extended position, where the energy storage member transitions from the stored state to the released state when fluid from the reservoir contacts the activation member.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings.

FIG. 1 is a perspective view of a drug delivery device according to one aspect or embodiment of the present application.

FIG. 2 is a perspective view of the drug delivery device of FIG. 1, with a top cover removed.

FIG. 3 is a schematic view of the drug delivery device of FIG. 1.

FIG. 4 is a schematic view of an activation member according to one aspect or embodiment of the present application, showing the activation member prior to device activation.

FIG. 5 is a schematic view of the activation member of FIG. 4, showing the activation member after device activation and subsequent activation of an insertion mechanism.

FIG. 6 is a schematic view of the activation member of FIG. 4, showing the activation member during infusion of medicament.

FIG. 7 is a schematic view of an activation member according to a further aspect or embodiment of the present application, showing the activation member prior to infusion of medicament.

FIG. 8 is a schematic view of the activation member of FIG. 7, showing the activation member after actuation of a drug delivery device.

FIG. 9 is a schematic view of the activation member of FIG. 7, showing the activation member during infusion of medicament.

FIG. 10 is a top view of the activation member of FIG. 7, showing the activation member after actuation of a drug delivery device.

FIG. 11 is a top view of the activation member of FIG. 7, showing the activation member during infusion of medicament.

FIG. 12 is a top view of the activation member of FIG. 7, showing the activation member during infusion of medicament.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

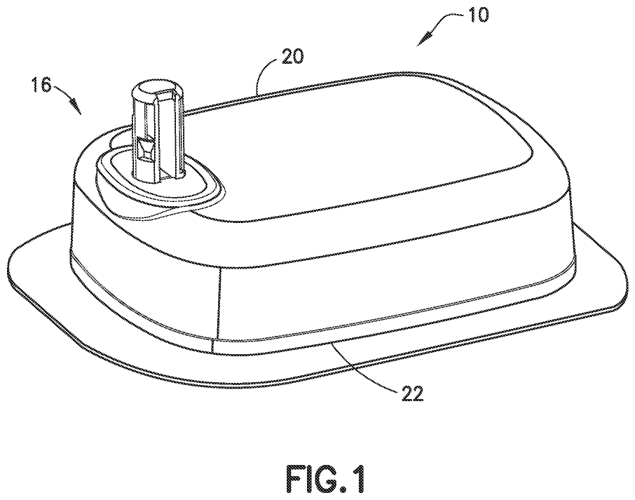

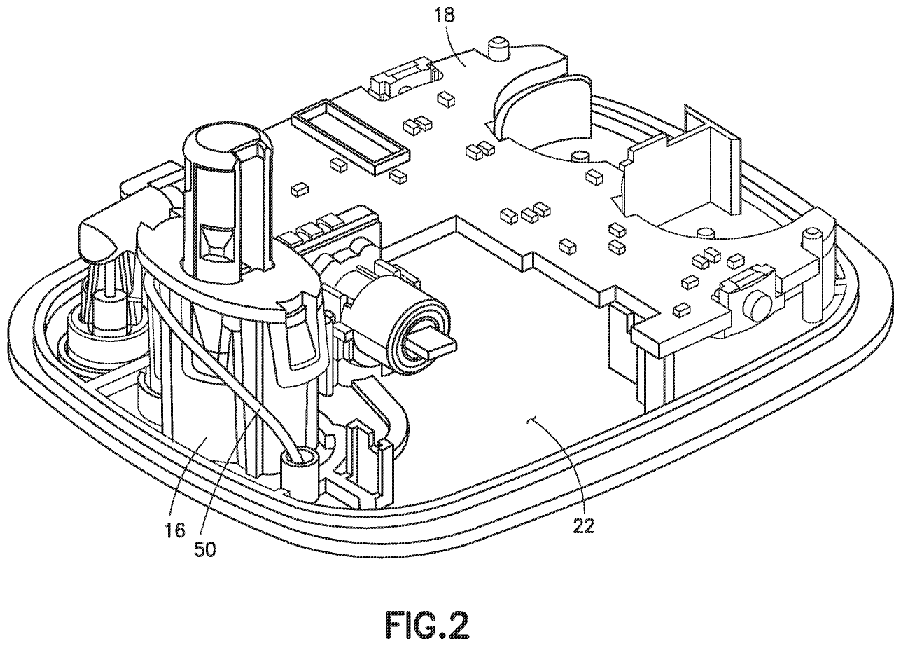

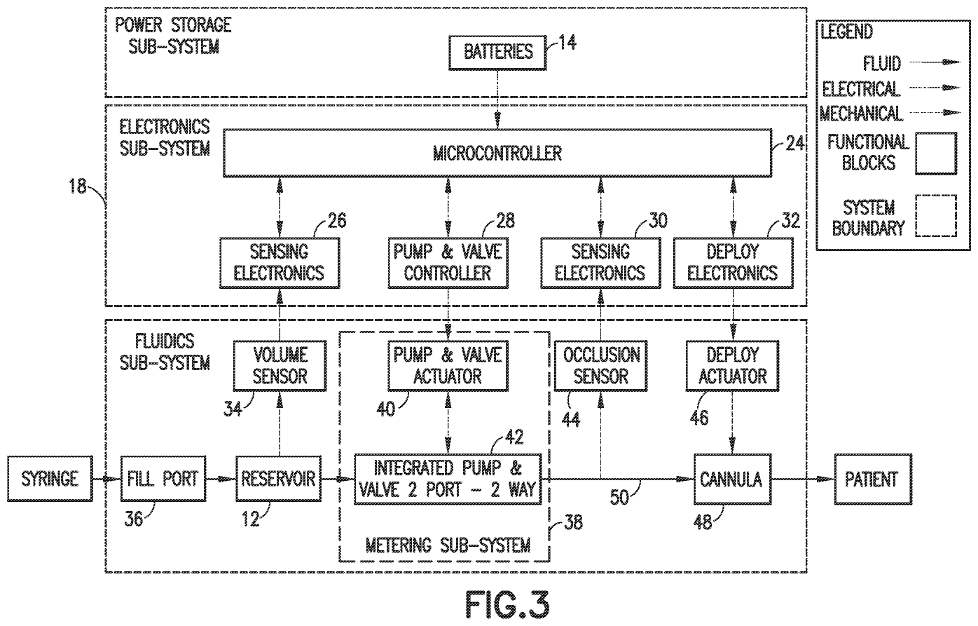

Referring to FIGS. 1-3, a drug delivery device 10 includes a reservoir 12, a power source 14, an insertion mechanism 16, control electronics 18, a cover 20, and a base 22. In one aspect or embodiment, the drug delivery device 10 is a wearable automatic injector, such as an insulin or bone marrow stimulant delivery device. The drug delivery device 10 may be mounted onto the skin of a patient and triggered to inject a pharmaceutical composition from the reservoir 12 into the patient. The drug delivery device 10 may be pre-filled with the pharmaceutical composition, or it may be filled with the pharmaceutical composition by the patient or medical professional prior to use.

The drug delivery device 10 is configured to deliver a dose of a pharmaceutical composition, e.g., any desired medicament, into the patient's body by a subcutaneous injection at a slow, controlled injection rate. Exemplary time durations for the delivery achieved by the drug delivery device 10 may range from about 5 minutes to about 60 minutes, but are not limited to this exemplary range. Exemplary volumes of the pharmaceutical composition delivered by the drug delivery device 10 may range from about 0.1 milliliters to about 10 milliliters, but are not limited to this exemplary range. The volume of the pharmaceutical composition delivered to the patient may be adjusted.

Referring again to FIGS. 1-3, in one aspect or embodiment, the power source 14 is a DC power source including one or more batteries. The control electronics 18 include a microcontroller 24, sensing electronics 26, a pump and valve controller 28, sensing electronics 30, and deployment electronics 32, which control the actuation of the drug delivery device 10. The drug delivery device 10 includes a fluidics sub-system that includes the reservoir 12, a volume sensor 34 for the reservoir 12, a reservoir fill port 36, and a delivery or metering sub-system 38 including a pump and valve actuator 40 and a pump and valve mechanism 42. The fluidic sub-system may further include an occlusion sensor 44, a deploy actuator 46, a cannula 48 for insertion into a patient's skin, and a fluid line 50 in fluid communication with the reservoir 12 and the cannula 48. In one aspect or embodiment, the insertion mechanism 16 is configured to move the cannula 48 from a retracted position positioned entirely within the device 10 to an extended position where the cannula 48 extends outside of the device 10. The cannula 48 may include a needle and/or catheter, with the needle piercing a patient's skin to place the catheter while subsequently retracting just the needle. The drug delivery device 10 may operate in the same manner as discussed in U.S. Pat. No. 10,449,292 to Pizzochero et al., incorporated herein by reference.

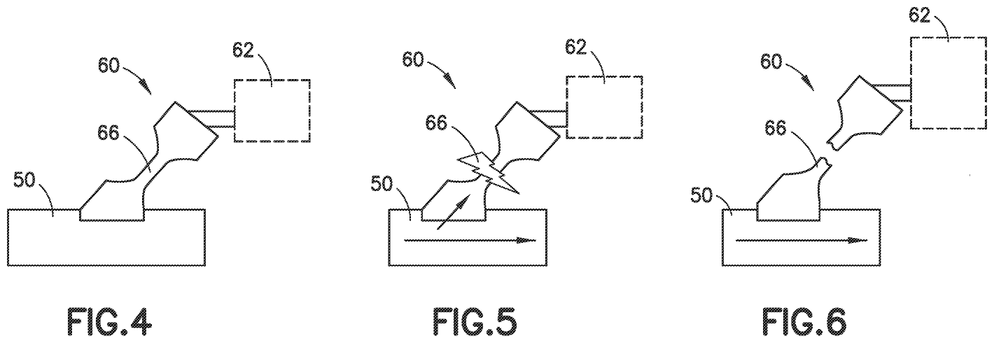

Referring to FIGS. 4-12, in one aspect or embodiment, the drug delivery device 10 includes an activation member 60 in fluid communication with the fluid path 50 and an energy storage member 62 connected to the activation member 60. The energy storage member 62 has a stored state when the cannula 48 is in the retracted position and a released state when the cannula 48 is in the extended position. The energy storage member 62 transitions from the stored state to the released state and completes insertion when fluid from the reservoir 12 contacts the activation member 60. Accordingly, the activation member 60 is configured to automatically move the cannula 48 from the retracted position to the extended position when the delivery sub-system 38 delivers fluid to the insertion mechanism 16. In some aspects or embodiments, the activation member 60 is configured to seal after coming into contact with fluid from the reservoir 12.

Referring to FIGS. 4-6, in one aspect or embodiment, the activation member 60 includes a hydrophilic material 66, with the hydrophilic material 66 having a first tensile strength when dry and a second tensile strength when wet. The first tensile strength is greater than the second tensile strength. Accordingly, when the hydrophilic material 66 is in contact with fluid from the reservoir 12, the weakening of the hydrophilic material 66 is configured to cause the energy storage member 62 to transition from the stored state to the released state, thereby moving the cannula 48 to the extended position. The hydrophilic material 66 prevents fluid from passing through the activation member 60 once the hydrophilic material 66 is fully saturated by fluid. In a further aspect or embodiment, the activation member 60 includes a dissolvable material positioned between absorbent materials, with the dissolvable material configured to disintegrate when in contact with a fluid.

As shown in FIG. 4, prior to activation of the drug delivery device 10, there is no fluid within the fluid path 50 and the energy storage member 62 is retained in the stored state by the activation member 60. As shown in FIG. 5, once the drug delivery device 10 is actuated, fluid flows from the reservoir 12 to the fluid path 50 and wets the activation member 60 which transitions the energy storage member 62 from the stored state to the released state, thereby moving the cannula 48 to the extended position. As shown in FIG. 6, after the activation member 60 is saturated, the activation member 60 is configured to seal to prevent further fluid flow through the activation member 60.

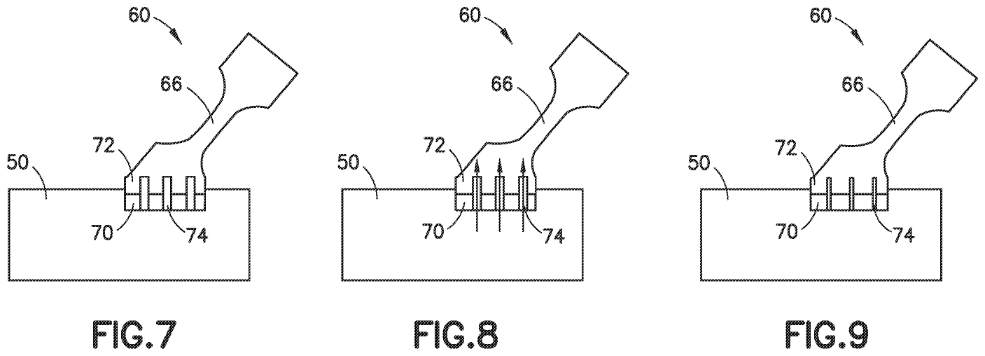

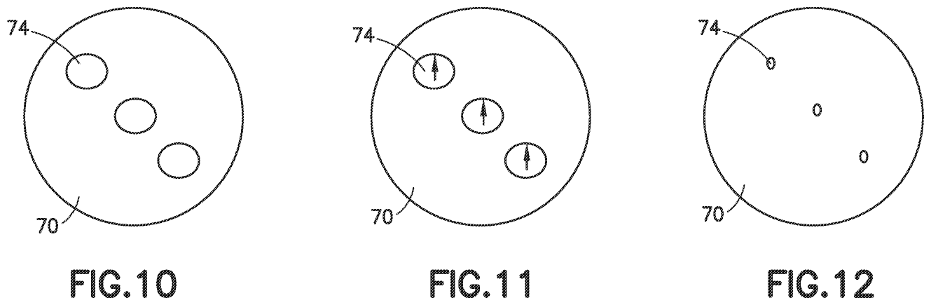

Referring to FIGS. 7-12, in a further aspect or embodiment, the activation member 60 further includes a hydrophobic layer 70 and a hydrophilic layer 72, with the hydrophobic layer 70 and the hydrophilic layer 72 defining a plurality of pores 74. The hydrophilic layer 72 is configured to expand, closing the plurality of pores 74, when the hydrophilic layer 72 is in contact with a fluid. As shown in FIGS. 7 and 10, prior to activation of the drug delivery device 10, there is no fluid within the fluid path 50 and the plurality of pores 74 remain open to allow fluid to contact the activation member 60. As shown in FIGS. 8 and 11, once the drug delivery device 10 is actuated, fluid flows from the reservoir 12 to the fluid path 50 and wets the activation member 60. As shown in FIGS. 9 and 12, when the hydrophilic layer 72 is saturated, the hydrophilic layer 72 expands to seal or close the plurality of pores 74 to prevent further fluid flow to the activation member 60. The hydrophobic layer 70 and the hydrophilic layer 72 are disc-shaped, although other suitable shapes and arrangements may be utilized. The plurality of pores 74 may be elliptical or circular, although other suitable shapes and arrangements may be utilized.

In one aspect or embodiment, the energy storage member 62 is a spring. The activation member 60 may be connected to the spring to retain the spring in a biased or compressed state, with the spring releasing when the activation member is in contact with a fluid.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. An insertion mechanism for a drug delivery device, the insertion mechanism comprising:
a fluid path configured to be in fluid communication with a reservoir;
an activation member in fluid communication with the fluid path; and
an energy storage member connected to the activation member, the energy storage member having a stored state and a released state, wherein the energy storage member transitions from the stored state to the released state when the fluid from the reservoir contacts the activation member.

2. The insertion mechanism of claim 1, wherein the activation member is configured to seal after coming into contact with the fluid from the reservoir.

3. The insertion mechanism of claim 1, wherein the activation member comprises a hydrophilic material, the hydrophilic material having a first tensile strength when dry and a second tensile strength when wet, and wherein the first tensile strength is greater than the second tensile strength.

4. The insertion mechanism of claim 3, wherein the hydrophilic material prevents fluid from passing through the activation member once the hydrophilic material is fully saturated by fluid.

5. The insertion mechanism of claim 1, wherein the activation member comprises a dissolvable material positioned between absorbent materials, the dissolvable material is configured to disintegrate when in contact with a fluid.

6. The insertion mechanism of claim 1, wherein the activation member comprises a hydrophobic layer and a hydrophilic layer, the hydrophobic layer and the hydrophilic layer define a plurality of pores, and wherein the hydrophilic layer is configured to expand closing the plurality of pores when the hydrophilic layer is in contact with a fluid.

7. The insertion mechanism of claim 1, wherein the energy storage member comprises a spring.

8. The insertion mechanism of claim 1, wherein the activation member comprises a dissolvable material configured to disintegrate when in contact with a fluid.

9. A drug delivery device comprising:
a housing;
a reservoir positioned within the housing and configured to receive a fluid;
a fluid path in fluid communication with the reservoir;
a delivery sub-system configured to deliver a fluid from the reservoir to the fluid path;
an insertion mechanism comprising a cannula in fluid communication with the fluid path, the insertion mechanism configured to move the cannula from a retracted position where the cannula is positioned within the housing to an extended position where at least a portion of the cannula is positioned outside of the housing;
an activation member in fluid communication with the fluid path; and
an energy storage member connected to the activation member, the energy storage member having a stored state when the cannula is in the retracted position and a released state when the cannula is in the extended position, wherein the energy storage member transitions from the stored state to the released state when the fluid from the reservoir contacts the activation member.

10. The device of claim 9 wherein the activation member is configured to seal after coming into contact with the fluid from the reservoir.

11. The device of claim 9, wherein the activation member comprises a hydrophilic material, the hydrophilic material having a first tensile strength when dry and a second tensile strength when wet, and wherein the first tensile strength is greater than the second tensile strength.

12. The device of claim 11, wherein the hydrophilic material prevents fluid from passing through the activation member once the hydrophilic material is fully saturated by fluid.

13. The device of claim 9, wherein the activation member comprises a dissolvable material positioned between absorbent materials, the dissolvable material is configured to disintegrate when in contact with a fluid.

14. The device of claim 9, wherein the activation member comprises a hydrophobic layer and a hydrophilic layer, the hydrophobic layer and the hydrophilic layer define a plurality of pores, and wherein the hydrophilic layer is configured to expand closing the plurality of pores when the hydrophilic layer is in contact with a fluid.

15. The device of claim 9, wherein the energy storage member comprises a spring.
